(19) United States
(12) Patent Application Publication
(10) Pub. No.: US 2007/0154534 A1
(43) Pub. Date: Jul. 5, 2007

(54) Use of secretin in the treatment of schizophrenia

(76) Inventors: Brian B. Sheitman, Cary, NC (US); Jeffrey A. Lieberman, Chapel Hill, NC (US); Michael B. Knable, Bethesda, MD (US)

Correspondence Address: JENKINS, WILSON, TAYLOR & HUNT, P. A., SUITE 1200, UNIVERSITY TOWER, 3100 TOWER BOULEVARD, DURHAM, NC, 27707, US

(21) Appl. No.: 10/548,685

(22) PCT Filed: Mar. 11, 2004
(86) PCT No.: PCT/US04/07304

§ 371 Date: Feb. 2, 2007

Related U.S. Application Data
(60) Provisional application No. 60/453,895, filed on Mar. 12, 2003.

(51) Int. Cl. A61K 38/54; A61K 9/70

(52) U.S. Cl. 424/449; 424/94.3

(57) ABSTRACT

The treatment of schizophrenia by administration of secretin to a patient in need thereof is disclosed.

### RELATED APPLICATIONS

This application claims the benefit of U.S. Provisional Patent Application Ser. No. 60/453,895, filed Mar. 12, 2003; the disclosure of which is incorporated herein by reference in its entirety.

### TECHNICAL FIELD

The present subject matter relates generally to therapeutic methods for mental disease. In one embodiment, the presently disclosed subject matter relates to the treatment of schizophrenia by administration of secretin to a patient in need of such treatment.

### Abbreviations

ANOVA Analysis of variance
CGI Clinical Global Impression Scale
CGI-I CGI Improvement Scale
CU Clinical units
DMSO dimethyl sulfoxide
DSM-IV Diagnostic and Statical Manual of Mental Disorders, Fourth Edition
GGA Geranyl-geranyl acetone
MCI-727 (Z)-2-(4-methylpiperazin-1yl)-1-[4-(2-phenyl -ethyl)phenyl]-eth anone oxime hydrochloride monohydrate
PANSS Positive and Negative Symptom Scale for Schizophrenia
PHP 1-phenyl-1-hydroxy-N-pentane
SMR Standardized mortality ratios
TPN tetraprenylacetone

### BACKGROUND ART

Schizophrenia is one of the most severe and debilitating of the major psychiatric diseases. It usually starts in late adolescence or early adult life and often becomes chronic and disabling. Men and women are at equal risk of developing this illness; however, most males become ill between 16 and 25 years old, while females develop symptoms between 25 and 30. People with schizophrenia often experience both “positive” symptoms (e.g., delusions, hallucinations, disorganized thinking, and agitation) and “negative” symptoms (e.g., lack of drive or initiative, social withdrawal, apathy, and emotional unresponsiveness). In addition, schizophrenia patients typically are afflicted with cognitive impairments, and deficits in social and vocational functioning.

Schizophrenia affects 1% of the world's population, an estimated 45 million people, with more than 33 million of them in the developing countries. This disease places a heavy burden on the patient's family and relatives, both in terms of the direct and indirect costs involved and the social stigma associated with the illness, sometimes over generations. Such stigma often leads to isolation and neglect.

Moreover, schizophrenia accounts for one fourth of all mental health costs and takes up one in three psychiatric hospital beds. Most schizophrenia patients are never able to be competitively employed. The cost of schizophrenia to society is enormous. In the United States, for example, the direct cost of treatment of schizophrenia has been estimated to be close to 0.5% of the gross national product. Standardized mortality ratios (SMRs) for schizophrenic patients are estimated to be two to four times higher than the general population, and their life expectancy overall is 20% shorter than for the general population. The most common cause of death among schizophrenic patients is suicide (in 10% of patients), representing a 20 times higher risk than for the general population. Deaths from heart disease and from diseases of the respiratory and digestive system are also increased among schizophrenic patients.

As there are currently no cures for schizophrenia, the objective of treatment is to reduce the severity of the symptoms, if possible to the point of remission. Antipsychotic medications are the most common treatments and are directed primarily against the positive symptoms of the illness. Chlorpromazine (thorazine) was the first antipsychotic introduced into clinical practice and is representative of the first generation of antipsychotics. This class of medication has the propensity to induce extra-pyramidal side effects (EPS) in most patients. This can result in permanent movement disorders (tardive dyskinesia) and other troublesome acute side effects (parkinsonian symptoms, and akathisia). The second generation antipsychotics, of which clozapine is the prototype, are somewhat more effective than the first generation drugs and cause much less EPS, but full remission is very rare and most patients remain severely impaired compared to those without the illness. In addition, the second-generation drugs often result in significant weight gain, dyslipidemia and glucose dysregulation.

Unfortunately, all the known drugs used for the treatment of schizophrenia have side effects and act only against some of the symptoms of the disease. Therefore, there is a strong need for new molecules that are more effective and without the associated side effects of current treatments

### SUMMARY

Disclosed is a method of treating schizophrenia and related disorders in a patient in need thereof, or an individual exhibiting one or more symptoms of schizophrenia, the method comprising administering an effective amount of secretin or a secretin analog to the patient.

In one embodiment, the secretin is synthetic, natural or recombinant secretin and is administered by intravenous, oral, intramuscular, sublingual, intra-articular, transdermal, subcutaneous, inhalation, or rectal administration. Further, the secretin can be human, porcine or bovine secretin.

In another embodiment, the secretin is administered with a transdermal carrier, for example, dimethyl sulfoxide. The transdermal carrier can comprise a gel or a lotion. The secretin and transdermal carrier can be mixed and applied to the skin together, or each applied to the skin separately. The secretin can be transdermally administered via a patch applied to the skin.

In one embodiment, the secretin is administered in an amount ranging from about 1 to about 500 clinical units (CU). In some embodiments, the secretin can be administered in an amount ranging from about 2 to about 200 CU, and in some embodiments, the secretin is administered in an amount ranging from about 75 to about 150 CU.

In another embodiment, the symptoms are positive symptoms, negative symptoms, affective symptoms, neurocognitive symptoms, social dysfunction, behavioral disorders and/or disorganized, compulsive, impulsive or repetitive behaviors.

Accordingly, it is an object of the presently disclosed subject matter to provide a novel therapeutic method for treatment of schizophrenia. This object is achieved in whole or in part by the methods disclosed herein and described below.

Objects of the subject matter disclosed herein having been stated above, other objects will become evident as the description proceeds when taken in connection with the accompanying Examples as best described herein below.

### DETAILED DESCRIPTION

I. Definitions

The term “about”, as used herein when referring to a value or to an amount of mass, weight, time, volume, or percentage is meant to encompass variations of ±20% or ±10%, more preferably ±5%, even more preferably ±1%, and still more preferably ±0.1% from the specified amount, as such variations are appropriate to perform the disclosed methods.

As used herein, an “effective” amount or dose refers to one that is effective or falls within an effective range in at least some of a population of patients and that is sufficient to modulate a condition and/or to cause an improvement in symptoms in a subject.

As used herein, “schizophrenia” refers to a serious, often chronic, mental disorder affecting a variety of aspects of behavior, thinking and emotion and as defined in the DSM-IV, including refractory schizophrenia. As used herein, the term “schizophrenia” is intended to encompass not only the specific disorder defined in the DSM-IV as schizophrenia, but also related mental disorders, including schizoaffective disorder and schizophreniform disorder. The phrase “treating a mental disease” is meant to refer to the treatment of mental disease at any stage of progression. Thus, treatment of early onset mental disease as well as treatment of advanced mental disease falls within the phrase “treating mental disease”. Preventing mental disease and/or reducing the severity of mental disease also fall within the phrase “treating mental disease”. In one embodiment the mental disease is schizophrenia. In another embodiment, the mental disease is refractory schizophrenia. In another embodiment, the mental disease is schizophreniform disorder. In yet another embodiment, the mental disease is schizoaffective disorder. “Treating” can mean the complete and permanent alleviation of symptoms of the mental disease, but is not limited to this definition. In one embodiment, “treating a mental disease” means a measurable reduction of the symptoms of the mental disease. In the case of schizophrenia and related disorders, the reduction in symptoms can be positive symptoms (e.g. active delusions, hallucinations, thought disorder, disorganized thinking and agitation), negative symptoms (e.g. lack of drive, lack of initiative, social withdrawal, apathy, emotional unresponsiveness, impaired social relations, affective flattening, alogia and avolition), affective symptoms, neurocognitive symptoms, social dysfunction, behavioral disorders and/or disorganized, compulsive, impulsive or repetitive behaviors. A reduction in symptoms can be determined by either experimentally and statistically relevant data or by clinical observation of the patient.

The subject treated in the many embodiments disclosed herein is desirably a human subject or patient, although it is to be understood that the principles of the presently disclosed subject matter indicate that the subject matter is effective with respect to all vertebrate species, including mammals, which are intended to be included in the terms “subject” and “patient”. In this context, a mammal is understood to include any mammalian species in which treatment is desirable, particularly agricultural and domestic mammalian species, such as horses, cows, pigs, dogs, and cats.

II. General Considerations

Secretin is a neuropeptide that stimulates excretion of water and bicarbonate from the pancreas and biliary tree and the secretion of digestive enzymes from the pancreas. It is secreted when the stomach empties. In the hypothalamus, two peptides known as hypocretins with substantial sequence homology to secretin have recently been described (de Lecea et al., 1998). Animal studies suggest that the hypocretins contribute to the regulation of sleep, arousal and motivation (Siegel, 1999).

Secretin is a 27-amino acid peptide hormone produced by the S-cells of the small intestine that are spatially distributed from the upper crypt to the villus tip, being particularly numerous in the upper two-thirds of the villi (Inokuchi et al. 1985). The release of secretin is increased by the products of protein digestion, acid bathing, fat, sodium-oleate, bile and herbal extracts (Leiter et al. 1994). Secretin increases the secretion of bicarbonate in the pancreas and biliary tract, resulting in secretion of a watery, alkaline pancreatic fluid. The effect of secretin on the pancreas and bile duct is mediated primarily by secretin-induced elevation of cyclic AMP (Lenzen et al. 1992), and does not involve the inositol phosphate signal transduction pathway.

Secretin regulates the growth and development (enzyme composition) of the stomach, small intestine, and pancreas, and stimulates pancreatic fluid secretion, and bile secretion (McGill et al. 1994). In addition, secretin has secretory, motility and circulatory effects in the gastrointestinal tract. Secretin stimulates immunoglobulin excretion through bile (Lebenthal and Clark 1981). Secretin increases superior mesenteric blood flow, and its distribution within the mucosa and submucosa (Fara and Madden 1975), as well as lymph flow (Lawrence et al. 1981).

Thus far, most clinical uses of secretin are based on its secretory and vascular effects. Two diagnostic applications for which secretin is used are the examination of pancreatic function, and the diagnosis of gastrinoma. A trial to use secretin in intrahepatic cholestasis in small numbers of patients initially was encouraging (Fukumoto et al. 1989); however, a double-blind placebo-controlled multicentric trial found no statistically significant differences in the reduction of serum bilirubin levels between secretin and placebo groups (Fukumoto et al. 1996). In the hypothalamus, two peptides known as hypocretins with substantial sequence homology to secretin have recently been described (de Lecea et al, 1998).

The structure and sequence of porcine secretin has been known for some time. It has been isolated from porcine intestine, and has been found to be constituted by a peptide composed of 27 amino acid residues (Mutt et al. 1970). Moreover, it has been found that bovine and porcine secretins are identical but that they are markedly different from chicken secretin (Carlquist et al. 1981).

Although bovine and porcine secretins behave identically with human secretin in some respects, they are not structurally identical. See, for example, U.S. Pat. No. 4,806,336 to Carlguist et al., incorporated by reference herein in its entirety, which discloses the chemical composition of human secretin, a method for administering secretin for diagnostic use in determining pancreatic or gallbladder function, and a method for stimulating pancreatic secretion in man.

III. Therapeutic Compounds

The methods disclosed encompass methods of treating schizophrenia and related disorders with secretin. Secretin, as used herein, refers to molecules possessing the biological activity of the natural human polypeptide. Therefore, “secretin” not only includes the human neuropeptide, but also animal secretins, equivalent analogs, biologically active fragments of the full-length secretin, and secretin fusion polypeptides. The analogs may be peptide or non-peptide analogs.

III.A. Secretin and Secretin Analogs

The presently disclosed subject matter includes use of human secretin, as well as homologous secretins, including bovine, porcine, equine, canine, and other mammalian secretins. The presently disclosed subject matter also includes the use of polypeptides that have a sequence substantially identical to secretin, e.g., secretin analogs. A polypeptide which is “substantially identical” to a given reference polypeptide is a polypeptide having a sequence that has at least 85% identity to the sequence of the given reference polypeptide sequence. Substantially identical polypeptides can also have a higher percentage identity, e.g., 90%, 95%, 98%, or 99%. The subject matter disclosed herein also encompasses polypeptides that are functionally equivalent to secretin. These polypeptides are equivalent to secretin in that they are capable of carrying out one or more of the functions of secretin in a biological system. Such polypeptides have 60%, 75%, 80%, or even 90% of one or more of the biological activities of full-length secretin. Such comparisons are generally based on an assay of biological activity in which equal concentrations of the polypeptides are used and compared. The comparison can also be based on the amount of the polypeptide required to reach 50% of the maximal stimulation obtainable.

Functionally equivalent polypeptides can be those, for example, that contain additional or substituted amino acid residues. Substitutions may be made on the basis of similarity in polarity, charge, solubility, hydrophobicity, hydrophilicity, and/or the amphipathic nature of the residues involved. For example, a functionally equivalent polypeptide is one in which 10% or fewer of the amino acids of full-length, naturally occurring secretin are replaced by conservative amino acid substitutions, and the functionally equivalent polypeptide maintains at least 50% of the biological activity of full-length secretin. Conservative amino acid substitution refers to the substitution of one amino acid for another amino acid of the same class (e.g., valine for glycine, or arginine for lysine). Polypeptides that are functionally equivalent to secretin can be made using random mutagenesis of the encoding nucleic acids by techniques well known to those skilled in the art. It is more likely, however, that such polypeptides will be generated by site-directed mutagenesis using techniques also well known to those skilled in the art. These polypeptides may have increased functionality or decreased functionality. To design functionally equivalent polypeptides, it is useful to distinguish between conserved positions and variable positions. This can be done by aligning the amino acid sequence of a protein of the presently disclosed subject matter from one species with its homolog from another species. Skilled artisans will recognize that conserved amino acid residues are more likely to be necessary for preservation of function. Thus, it is preferable that conserved residues are not altered.

Mutations within the coding sequence of a nucleic acid molecule encoding secretin can be made to generate variant genes that are better suited for expression in a selected host cell. For example, N-linked glycosylation sites can be altered or eliminated to achieve, for example, expression of a homogeneous product that is more easily recovered and purified from yeast hosts that are known to hyperglycosylate N-linked sites. To this end, a variety of amino acid substitutions at one or both of the first or third amino acid positions of any one or more of the glycosylation recognition sequences which occur, and/or an amino acid deletion at the second position of any one or more of such recognition sequences, will prevent glycosylation at the modified tripeptide sequence (see, for example, Miyaiima et al.).

III.B. Secretin Fusion Proteins

The polypeptide secretins and analogs used herein can also be expressed fused to another polypeptide, for example, a marker polypeptide or fusion partner. For example, the polypeptide can be fused to a hexa-histidine tag to facilitate purification of bacterially expressed protein or a hemagglutinin tag to facilitate purification of protein expressed in eukaryotic cells. A fusion protein may be readily purified by utilizing an antibody specific for the fusion protein being expressed. For example, a system described by Janknecht et al. (1991) allows for the ready purification of non-denatured fusion proteins expressed in human cell lines. In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺ nitriloacetic acid-agarose columns and histidine-tagged proteins are selectively eluted with imidazole-containing buffers.

The polypeptide secretin analogs used as part of the presently disclosed subject matter can also be chemically synthesized (for example, see Creighton, 1983), or, perhaps more advantageously, produced by recombinant DNA technology. For additional guidance, skilled artisans may consult Sambrook et al., and, particularly for examples of chemical synthesis (Gait, M.J. (Ed.)).

III.C. Nonpeptide Secretin Analogs

In addition to polypeptide secretin analogs, nonpeptide analogs can also be used with the subject matter disclosed herein. These nonpeptide analogs can include any small molecule that shows an activity equivalent to secretin. Such analogs can be generated, for example, by combinatorial chemical techniques that optimize their secretin-like activity.

IV. Therapeutic Methods

A method of treating a mental disease by administration of an effective amount of secretin to a patient in need thereof is disclosed. In one embodiment the mental disease is schizophrenia. In another embodiment, the mental disease is refractory schizophrenia. In another embodiment, the mental disease is schizophreniform disorder. In yet another embodiment, the mental disease is schizoaffective disorder. Treating the mental disease can comprise at least partially alleviating symptoms of the disease, at least transiently. Representative symptoms that can be alleviated include but are not limited to positive symptoms (e.g. active delusions, hallucinations, thought disorder, disorganized thinking and agitation), negative symptoms (e.g. lack of drive, lack of initiative, social withdrawal, apathy, emotional unresponsiveness, impaired social relations, affective flattening, alogia and avolition), affective symptoms, neurocognitive symptoms, social dysfunction, behavioral disorders and/or disorganized, compulsive, impulsive or repetitive behaviors. Symptoms alleviated can be all or some of the symptoms.

IV.A. Dosages

Optionally, the secretin is chosen from the group consisting of human secretin, porcine secretin, and bovine secretin. Also optionally, the secretin is synthetic secretin, natural secretin, or recombinant secretin. Secretin is commercially available under the trademark SECREFLO™ from RepliGen Corporation of Waltham, Mass., United States of America, and as SECRETIN-FERRING™ from Ferring Laboratories, Inc., Suffern, N.Y., United States of America. The SECREFLO™ product demonstrates a potency of approximately 5000 clinical units (CU) per milligram of peptide as opposed to 3000 CU per milligram for biologically derived porcine secretin. The relationship of micrograms of secretin to biological activity in clinical units is 0.2 micrograms (mcg)=1 CU.

The secretin can be administered in an effective amount over any desired period of time, e.g. daily over a desired period of time, such as two days, three days, four days, five days, or longer; weekly over a desired period of time, such as once, twice, or three times a week for one week, two weeks, three weeks, four weeks, or longer; or monthly over a desired period of time, such as once or twice a month, for one month, two months, three months, or longer. In treatment regimes of longer durations between administration, sustained released formulations can be used. In another embodiment, due to the chronic nature of schizophrenia, the effective amount is administered in a regular pattern over an indefinite period of time to control symptoms.

Several dosage options and approaches are provided. In one embodiment, the secretin is administered in an amount ranging from about 1 to about 500 CU, optionally about 2 to about 200 CU, optionally about 10 to about 150 CU, including about 20, 25, 50, 75 and 100 CU. Alternatively, the secretin is administered in an amount ranging from about 0.1 micrograms per kilogram to about 10 micrograms per kilogram of the patient's body weight, optionally about 0.2 to about 5 microgram per kilogram body weight, including 0.3, 0.4, 0.5, 0.6, 0.7, 0.8, 0.9, 1.0, and 2.5 microgram per kilogram body weight.

IV.B. Administration

Provided is a novel, effective dose of secretin (in one embodiment, the secretin is administered intravenously) that alleviates one or more symptoms of schizophrenia in certain individuals suffering from schizophrenic syndromes. Additionally, oral, intramuscular, intra-articular, transdermal, subcutaneous, inhalation, and rectal routes of administration can be effective.

To elaborate, in one embodiment intravenous administration of a bolus of secretin in solution is provided. However, alternate, less-invasive, routes of secretin application from external sources, such as rectal and transdermal routes, are also provided herein. As is known in the art, such administration typically requires attachment of certain biologically compatible chemicals to assist in the mucosal or dermal absorption (know as permeation enhancers) and to protect against hydrolysis by the colonic bacterial flora or other cellular enzymes.

In another example, secretin is administered transdermally by applying dimethyl sulfoxide (DMSO) to the patient's skin and rubbing an effective amount of the secretin into the DMSO. Pharmaceutical grade DMSO (generally 99.9% pure) is available from Clinic Service Company, Hemet, Calif., United States of America. By way of example for each treatment, about four drops of DMSO are placed onto the skin of the patient, an effective amount of secretin is placed onto the DMSO, and the composition is rubbed into the skin.

Other methods and compositions for administering the effective amount of secretin include other transdermal carrier substances, such as gels, lotions, or patches; oral carriers, such as tablets, capsules, or lozenges; inhalation through the nose or mouth (e.g., as an aerosol); suppository forms of secretin and secretin compositions; intravenous administration; and using acoustic waves to cause the secretin to penetrate the skin.

Also provided is the use of other types of transdermal carrier substances in addition to DMSO. Other alternative ways of administering secretin include, but are not limited to, administering secretin transdermally with a gel, lotion or patch; administering secretin with a suppository; administrating secretin orally, as tablet, capsule or lozenge; administrating secretin by inhalation (e.g., as an aerosol) either through the mouth or the nose. Such alternative methods of administering secretin are less invasive, do not have to be carried out by a doctor at a medical facility, and are less expensive. In addition, the level or dose of administration of secretin can be varied from those examples stated herein including, for example, intravenous administration over a period of time of several hours instead of several minutes and/or a smaller, maintenance or daily dose administered intramuscularly, transdermally or by other methods as disclosed herein or their equivalent.

A further alternative method of transdermally administering secretin includes the use of acoustic waves to permeate the skin. For example, acoustic waves generated using ultrasound or a shockwave from a pulsed laser have been found to make the skin temporarily permeable. A few minutes of low-frequency ultrasound (sound greater in frequency than 20 kilohertz) creates tiny cavities through which the secretin (alone or combined with another transdermal carrier substance) can be diffused.

Additional formulation preparation techniques have been generally described in the art, see for example, those described in U.S. Pat. No. 5,326,902 issued to Seipp et al. on Jul. 5, 1994, U.S. Pat. No. 5,234,933 issued to Marneft et al. on Aug. 10, 1993, and PCT Publication WO 93/25521 of Johnson et al. published Dec. 23, 1993, and each of which is herein incorporated by reference in its entirety.

For the purposes described above, the therapeutic agent (e.g. secretin) can normally be administered systemically or partially, usually by oral or parenteral administration. The doses to be administered are determined depending upon age, body weight, symptom, the desired effect, the route of administration, and the duration of the treatment, etc. One of skill in the art of therapeutic treatment will recognize appropriate procedures and techniques for determining the appropriate dosage regimen for effective therapy. Additional guidance is also provided in the Examples set forth herein. Various compositions and forms of administration are provided and are generally known in the art. Other compositions for administration include suppositories that comprise one or more of the active substance(s) and can be prepared by known methods.

Alternate approaches for increasing secretin levels in the body are also included in the methods disclosed herein. In one embodiment, stimulating secretin release is also provided herein and can be used in the new methods. For example, certain agents when delivered orally cause the body to release secretin, e.g., as described in U.S. Pat. Nos. 6,197,746 to Beech et al. and 6,498,143 to Beech et al., incorporated herein by reference in their entireties. By way of particular example, studies have shown that a decrease in the pH of the duodenum below 4.5 results in a significant secretin release. Administration of hydrochloric acid has been shown not only to stimulate the release of secretin but also to stimulate the biosynthesis of secretin (Murthy, 1981). Likewise, gastric acids can trigger the release of secretin. Therefore, it is clear that exogenous administration or endogenous production of acidic agents can lead to the release of secretin as well as the endogenous production of the hormone.

Other agents linked to secretin production and/or release include but are not limited to 1-phenylpentanol or 1-phenyl-1-hydroxy-N-pentane (PHP); bile salts and acids; fats and fatty acids such as sodium oleate and oleic acid; anti-ulcer compounds such as PLAUNOTOL™, tetraprenylacetone (TPN), geranyl-geranyl acetone (GGA), and (Z)-2-(4-methylpiperazin-1yl)-1 -[4-(2-phenyl-ethyl)phenyl]-eth anone oxime hydrochloride monohydrate (MCI-727); and herbal extracts such as licorice root. Thus, it is within the scope of the presently disclosed subject matter to exogenously administer a substance that can either stimulate the release of secretin or stimulate the endogenous production of the hormone.

IV.C. Formulations

A pharmaceutical composition in accordance with the presently disclosed subject matter can be formulated with one or more physiologically acceptable carriers or excipients. Furthermore, in some embodiments, compositions can comprise more than one active agents. For example, a composition can include secretin as well as one or more other agents also effective at treating schizophrenia and related disorders. In some embodiments several active ingredients can be formulated in one composition for simultaneous delivery to a patient, whereas in other embodiments the active ingredients are formulated in separate compositions to facilitate delivery at different times to a patient, at different sites on a patient, at different dosage levels, or by different routes (e.g. oral and transdermal, etc.).

The compounds for use according to the present subject matter can be formulated for oral, buccal, sublingual, parenteral, rectal or transdermal administration, or administration in a form suitable for inhalation or insufflation (either through the mouth or the nose). In one embodiment a transdermal patch is employed. In another embodiment an oral preparation is employed. In another embodiment, an injection that has long term benefits is employed, e.g. a sustained release formulation. Administration can also be accomplished by any other effective techniques.

For oral administration, the pharmaceutical compositions can take the form of, for example, tablets or capsules prepared by a conventional technique with pharmaceutically acceptable excipients such as binding agents (e.g. pregelatinized maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium starch glycolate); or wetting agents (e.g. sodium lauryl sulphate). The tablets can be coated by methods well known in the art.

Liquid preparations for oral administration can take the form of, for example, solutions, syrups or suspensions, or they can be presented as a dry product for constitution with water or other suitable vehicle before use. Such liquid preparations can be prepared by conventional techniques with pharmaceutically acceptable additives such as suspending agents (e.g. sorbitol syrup, cellulose derivatives or hydrogenated edible fats); emulsifying agents (e.g. lecithin or acacia); non-aqueous vehicles (e.g. almond oil, oily esters, ethyl alcohol or fractionated vegetable oils); and preservatives (e.g. methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound. For buccal administration the compositions can take the form of tablets or lozenges formulated in a conventional manner.

The methods of administration according to the presently disclosed subject matter can include parenteral administration by injection, for example by bolus injection or continuous infusion. Formulations for injection can be presented in unit dosage form, e.g., in ampoules or in multi-dose containers, with or without an added preservative. An injectable formulation can be used in delivering a therapeutic agent across the blood brain barrier to the central nervous system.

The compositions used in the methods can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g. sterile pyrogen-free water, before use. The compounds can also be formulated in rectal compositions such as suppositories or retention enemas, e.g. containing conventional suppository bases such as cocoa butter or other glycerides. In addition to the formulations described previously, the compounds can also be formulated as a preparation for implantation or injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

### EXAMPLES

The Examples have been included to illustrate representative modes of the presently disclosed subject matter. In light of the present disclosure and the general level of skill in the art, those of skill will appreciate that the following Examples are intended to be exemplary only and that numerous changes, modifications and alterations can be employed without departing from the spirit and scope of the presently disclosed subject matter.

### A. Summary of Examples

A pilot study of secretin for treatment of refractory schizophrenia was performed. Twenty-two patients were randomized to a single intravenous dose of porcine secretin or placebo. Patients were evaluated with the Positive and Negative Symptom Scale for Schizophrenia (PANSS) and the Clinical Global Impression Scale (CGI) at baseline, two days after secretin infusion and weekly for four weeks. Several patients treated with secretin experienced clinically meaningful, though transient when given a single dose, reductions in symptoms and a greater percentage of patients treated with secretin were rated as improved with the CGI.

### B. Methods of Examples

All subjects were inpatients at Dorothea Dix Hospital in Raleigh, N.C. United States of America. The protocol was approved by the University of North Carolina at Chapel Hill Committee for the Protection of the Rights of Research Subjects. Subjects or their guardians provided written informed consent. Patients met the following inclusion criteria: (1) age 18-60; (2) DSM-IV diagnosis of schizophrenia or schizoaffective disorder; (3) severe residual symptoms despite at least three adequate trials of anti-psychotic drugs, including at least one atypical drug. Residual symptoms were defined as active delusions, hallucinations, or thought disorder, accompanied by unemployment and lack of significant social relationships outside the family of origin; (4) a baseline PANSS score of at least 70; (5) no changes in psychotropic medications for 4 weeks prior to study entry. Baseline demographic and clinical characteristics of the subjects are displayed in Table 1. Concurrent anti-psychotic medications and other classes of psychotropics at time of study entry are listed in Table 2.

Twenty-two subjects were enrolled in the protocol and were randomly assigned to receive one dose of intravenous porcine secretin (SECRETIN-FERRING™, Ferring Laboratories, Inc., Suffern, N.Y., United States of America) or normal saline under double-blind conditions. The protocol allowed for only one infusion of secretin regardless of response. The first seven subjects received either 75 CU of secretin dissolved in 7.5 mL of normal saline (n=4), or saline alone (n=3). The remaining 15 subjects received 150 CU of secretin (n=7) or saline (n=8). The decision to increase the dose during the study was made after a clinically meaningful, though transient improvement was observed in subject #7. By increasing the dose of secretin the objective was to see if an increased percentage of clinically meaningful improvements in the remaining subjects would be observed, and to assess if the improvement would persist for a longer duration. In addition, there had been no side effects noted in the first seven subjects at the 75 CU dose.

All subjects prior to the infusion had an intravenous line started with normal saline at a rate intended to keep the intravenous line running. Next subjects received a 0.1 mL intravenous push of a clear solution of either active medication or placebo, and then were observed for 10 minutes for the occurrence of an allergic reaction. If no allergic reaction occurred then the remaining 7.4mL of solution was infused over the next 60 seconds by an intravenous push (no allergic reactions were observed in any of the subjects).

Subjects were assessed at baseline, two days post-infusion, and weekly for four subsequent weeks, with the Clinical Global Impression Scale (CGI) and the Positive and Negative Symptom Scale for Schizophrenia (PANSS). All raters achieved inter-rater reliability of at least 0.90 for the PANSS, and each subject had the same rater for each assessment. Qualitative impressions were also collected from clinical staff during the course of the trial.

### C. Subject Examples

At baseline there were no significant differences between the drug-treated and placebo-treated subjects for age, gender, race, illness duration, PANSS negative symptom scores, PANSS general psychopathology scores or CGI scores. The drug treated group was significantly more ill than the placebo group at baseline for total PANSS scores and for PANSS positive symptom scores (p=0.03). There were no premature dropouts or serious adverse events during the trial.

Using repeated measures ANOVA with treatment status as a between group factor, and time as a within-group factor, there were no significant group by time interactions for PANSS total scores (F=1.72, df=20,80), PANSS positive symptom scores (F=1.08), PANSS negative symptom scores (F=0.6679), or PANSS general psychopathology scores (F=0.41). Similarly, no significant differences were found between the groups when baseline differences in disease severity were controlled for with analysis of covariance, and there was no significant group by time interaction for the CGI-Improvement (CGI-I) scale (F=0.69). Table 3 displays the PANSS and CGI-I scores during the course of the trial.

However, patients that received secretin were consistently rated as clinically more improved than those treated with placebo. Comparing the groups with Student's t-tests in a post-hoc exploratory fashion revealed significant improvement in the drug treated patients two days after infusion (t=−2.415, p=0.03), at week 1 (t=−2.899, p=0.009) and at week 3 (t=−3.652, p=0.002) on the CGI-I scale. Improvement was also noted when a qualitative grouping of CGI-I scores was performed. For example, amongst the secretin-treated patients, three were rated as much improved, five were minimally improved, two were unchanged, and one was worse within the first week after infusion. Amongst the placebo-treated patients, none were rated as much improved, one was minimally improved, seven were unchanged, one was minimally worse, and two were much worse. These results are particularly significant considering all the patients chosen for this study were severely ill and had not responded to any of previous lengthy treatments with many types of traditional treatments. Therefore, even though the patients were unresponsive to numerous traditional therapies, treatment with secretin provided clinically significant reductions in one or more symptoms of schizophrenia.

Increasing the dose of medication infused did not substantially increase the percentage of patients with a clinically meaningful response (1 of 4, versus 2 of 7), nor did the duration of the response time increase.

### Example 1

The first of the three patients who were rated as much improved as demonstrated by a clinically relevant, though transient improvement in symptoms received the 75 CU dose of secretin. He is a 43 year-old male with disorganized schizophrenia for over 25 years who had rarely lived outside of the hospital. He typically remained alone and played obsessionally with toys meant for young children. A combination of fluphenazine 20 mg/day and quetiapine 400 mg/day for six months prior to study entry had been ineffective. Within 24 hours of the secretin infusion the patient was more interactive. He approached a nurse and inquired about her pregnancy. He asked other patients to join him for board games, volunteered to clean up the ward, and reported to his psychiatrist that he felt “much better”. After four days he had reverted to his usual behavior and level of function.

### Example 2

A 45 year-old male with disorganized schizophrenia had been ill for over 30 years, and had spent the last 15 years as an inpatient received the 150 CU dose of secretin. He had severe thought disorder, usually at the level of “word salad”, and persistent delusions. He had received clozapine 600 mg/day and valproic acid for over two years prior to study entry. Within six hours of the secretin infusion, staff felt the patient was more alert. He was able to use coherent, grammatical sentences. Nursing staff reported that they had rarely heard comprehensible speech from him before. Therefore, although his symptoms had proven refractory to all previous traditional therapies, within only a few hours of treatment with a single dose of secretin, a clinically significant reduction in positive symptoms was noted. Although, the benefits were transient, and he seemed to return to his baseline mental state within 12 hours, it is possible that sustained treatment with secretin could provide prolonged relief from symptoms.

### Example 3

A 35 year-old female with paranoid schizophrenia had been ill for 19 years, and had been an inpatient for the eight years prior to study entry also received the 150 CU dose of secretin. She had severe terrifying, delusions that prevented her from leaving the inpatient ward. She had been receiving clozapine 300 mg/day for over six months at study entry without a significant control of her symptoms. Notably, 48 hours after secretin infusion she agreed to attend off ward activities. She had not done so except for a very brief trial approximately four months earlier. Though still delusional, she was less afraid and reported a feeling of relaxation. This lasted for about five days when she again became intensely fearful and refused to attend off ward activities. As with the patient in Example 2, although her symptoms had proven refractory to traditional therapy, a single dose of secretin had successfully alleviated some of her symptoms, including positive symptoms, and that relief was maintained for five days after cessation of treatment.

### D. Discussion of Examples

The Examples disclose that several patients who received secretin infusions experienced clinically relevant improvements in symptoms. A greater percentage of secretin-treated patients were rated as improved on the CGI-I compared to those who received placebo.

The observation of improvement is noteworthy. Several aspects of this particular patient cohort could have produced a bias towards a negative result. First, the patients enrolled in the study were severely ill and had been refractory to lengthy trials of many types of traditional treatments. Second, the randomization scheme was not optimal; the patients randomized to secretin were more ill at baseline than placebo patients. Third, only a single dose of secretin was administered. The present results thus support the use of secretin in the treatment of schizophrenia, a disease for which current treatments are often unsatisfactory.

TABLE 1
Baseline demographic and clinical characteristics.

| | |
|---|---|
| SECRETIN | PLACEBO |

| N | 11 | | 11 | |
|---|---|---|---|---|
| Age | 41.5 | (8.6) | 42.0 | (11.9) |
| Gender | 10M/1F | | 10M/1F | |
| Race | 8C/3AA | | 7C/4AA | |
| Diagnosis | | | | |
| Undifferentiated | 7 | | 6 | |
| Disorganized | 2 | | 1 | |
| Schizoaffective | 1 | | 2 | |
| Paranoid | 1 | | 2 | |
| Illness duration | 22.5 | (6.7) | 19.9 | (12.4) |
| Total PANSS* | 101.2 | (16.8) | 87.4 | (10.4) |
| PANSS positive* | 27 | (4.7) | 22.4 | (4.8) |
| PANSS negative | 25.5 | (6.3) | 22.3 | (4.8) |
| PANSS general | 48.7 | (8.9) | 42.7 | (6.8) |
| CGI-S | 5.6 | (0.92) | 4.9 | (0.9) |

*The drug and placebo-treated groups were significantly different at baseline (p < 0.03).

TABLE 2
Anti-psychotic Medications

| | SECRETIN¹ | PLACEBO² |
|---|---|---|
| Clozapine | 7 | 5 |
| Risperdone | 1 | 1 |
| Olanzapine | 1 | 3 |
| Typicals | 2 | 2 |

¹Two clozapine treated patients received monotherapy. Of the other five, four were prescribed a mood stabilizer, and three antidepressants. The risperidone treated patient was prescribed an antidepressant, the olanzapine patient a mood stabilizer. Of the two patients treated with a typical antipsychotic, one was also prescribed quetiapine.
²Two clozapine treated patients received monotherapy. Of the other three, all were prescribed mood stabilizers, two also received a typical antipsychotic. The risperidone treated patient received two mood stabilizers. One olanzapine patient was prescribed an antidepressant. Both patients on typicals received long term injections, one also received a mood stabilizer.

TABLE 3
PANSS and CGI-I scores.

| | Baseline | | Day 2 | Week 1 | | Week 2 | | Week 3 | | Week 4 | |
|---|---|---|---|---|---|---|---|---|---|---|---|
| Secretin | | | | | | | | | | | |
| PANSS total | 101.2 | (16.8) | | 95.1 | (13.7) | 100.8 | (12.5) | 100.1 | (14.5) | 99.5 | (16.4) |
| PANSS positive | 27 | (4.7) | | 25 | (5.5) | 26.9 | (3.4) | 27 | (4.7) | 26.7 | (4.5) |
| PANSS negative | 25.5 | (6.3) | | 24.3 | (5.7) | 24.7 | (5.7) | 24.9 | (6.1) | 24.5 | (5.7) |
| PANSS general | 48.7 | (8.9) | | 45.8 | (8.8) | 49.2 | (7.2) | 48.2 | (7.4) | 48.3 | (8.9) |
| CGI-I | | | 3.5 (0.8)* | 3.4* | (0.8) | 3.9 | (1.5) | 3.8* | (0.4) | 3.9 | (0.7) |
| Placebo | | | | | | | | | | | |
| PANSS total | 87.4 | (10.9) | | 88.5 | (10.9) | 89.3 | (13.6) | 90.4 | (12.5) | 88.5 | (15.2) |
| PANSS positive | 22.4 | (4.8) | | 23.1 | (5.9) | 22.6 | (5.4) | 23.5 | (5.3) | 22.6 | (5.7) |
| PANSS negative | 22.3 | (4.9) | | 22.7 | (6.0) | 21.7 | (5.2) | 23.6 | (5.2) | 22.5 | (5.4) |
| PANSS general | 42.7 | (6.8) | | 42.7 | (8.0) | 44.9 | (10.1) | 43.3 | (6.7) | 43.4 | (7.9) |
| CGI-I | | | 4.1 (0.3) | 4.4 | (0.8) | 4.2 | (0.9) | 4.5 | (0.5) | 4.3 | (0.8) |

*Post hoc Student's t-tests revealed significant improvement in the secretin treated patients two days after infusion (t = −2.415, p = 0.03), at week 1 (t = −2.899, p = 0.009) and at week 3 (t = −3.652, p = 0.002) on the CGI-I scale. There were no statistically significant differences between groups on any of the PANSS measures.

### REFERENCES

The references listed below as well as all references cited in the specification are incorporated herein by reference to the extent that they supplement, explain, provide a background for or teach methodology, techniques and/or compositions employed herein.

Bachus S E, Hyde T M, Herman M H, Egan M F, Kleinman J E, J Psychiatr Res., 1997; 31: 233-256.

Carlquist M, Jornvall H, Mutt V, FEBS Letters, 1981; 127(1 ):71-4.

Coniglio S J, Lewis J D, Lang C, Burns T G, Subhani-Siddique R, Weintraub A, Schub H, Holden E W, J Pediatr., 2001; 138: 649-655.

Creighton, Proteins: Structures and Molecular Principles, W. H. Freeman & Co., NY, 1983.

Diagnostic and Statistical Manual of Mental Disorders, Fourth Edition, (DSM IV), American Psychiatric Association.

DeLecea L, Kilduff T S, Peyron C, Gao X-B, Foye P E, Danielson P E, Fukuhara C, Battenberg E L F, Glautvik V T, Bartlett F S, Frankel W N, van den Pol A N, Bloom F E, Gautvik K M, Sutcliffe J G, Proc Nati Acad Sci USA, 1998; 95: 322-327.

Dunn-Geier J, Ho H H, Auersperg E, Doyle D, Eaves L, Matsuba C, Orrbine E, Pham B, Whiting S, Dev Med Child Neurol, 2000; 42: 796-802.

Fara J W, Madden K S, Am J Phys, 1975; 229(5):1365-70.

Fukumoto Y, Okita K, Yasunaga M, et al., Gastroenterologia Japonica, 1989; 24(3): 298-307.

Fukumoto Y, Okita K. Kodama T., J Gastroenterology, 1996; 31 (3): 394-403.

Gabriel S M, Davidson M, Haroutunian V, Powchik P, Bierer L M, Purohit D P, Perl D P, Davis K L,. Biol Psychiatry, 1996; 39: 79-81.

Gait, M.J. Ed., Oligonucleotide Synthesis, IRL Press, Oxford, 1984.

Horvath K, Stefanatos G, Sokolski K N, Wachtel R, Nabors L, Tildon J T,. J Assoc Acad Minor Phys, 1998; 9: 9-15.

Inokuchi H. Fujimoto S, Hattori T, Kawai K, Gastroenterology, 1985; 89(5): 1014-20.

Janknecht R, de Martynoff G, Lou J, Hipskind R A, Nordheim A, and Stunnenberg H G, Proc. Nati. Acad. Sci. USA, 1991; 88: 8972-8976.

Lahti R A, Cochrane E V, Roberts R C, Conley R R, Tamminga C A, J Neural Transm, 1998;105: 507-516.

Lawrence J A, Bryant D, Roberts K B, Barrowman J A, Quarterly J Exp Phys, 1981; 66(3): 297-305.

Lebenthal E, Clark B, Am J Gastroenterology, 1981; 75(6): 436-9.

Leiter A B. Chey W Y, Kopin A S. “Secretin” in Gut peptides: Biochemistry and Physiology edited by J H Walsh and G J Dockray, Raven Press, Ltd. New York., 1994:147-93.

Lenzen R, Alpini G, Tavoloni N, Am J Phys, 1992; 263(4 pt 1): G527-32.

Lightdale J R, Hayer C, Duer A, Lind-white C, Jenkins S, Siegel B, Elliot G R, Heyman M B, Pediatrics, 2001;. 108: 90.

McGill J M, Basavappa S, Geftys T W, Fitz J G, Am J Phys, 1994; 266(4 pt 1): G731-6.

Moroji T, Watanabe N, Aoki N, Itoh S, Arch Gen Psychiatry, 1982; 39(4): 485-6.

Miyajima A, Otsu K, Schreurs J, Bond M W, Abrams J S, Arai K, EMBO J., 1986; 5:1193-7.

Murthy, Gastroenterology, 1981; 80:1237.

Mutt V, Jorpes J E, Magnusson S, Eur J Biochem, 1970; 15(3): 513-9.

Nair N P V, Bloom D M, Nestoros J N, Schwartz G, Psychopharmacol Bull, 1983; 19: 134-136.

Owley T, McMahon W, Cook E H, Laulhere T, South M, Mays L Z, Shernoff E S, Lainhart J, Modahl C B, Corsello C, Ozonoff S, Risi S, Lord C, Leventhal B L, Filipek P A, J Am Acad Child Adolesc Psychiatry,2001; 40: 1293-1299.

Pollack P F. Wood J G, Solomon T, Digestive Dis & Sci, 1990; 35(6): 749-58.

Roberts W, Weaver L, Brian J, Bryon S, Emelianova S, Griffiths A M, MacKinnon B, Yim C, Wolpin J, Koren G, Pediatrics, 2001; 107: E71.

Sambrook et al. Molecular Cloning, A Laboratory Manual, Cold Spring Harbor Press, Cold Spring Harbor, N.Y., 1989.

Sandier A D, Sutton K A, DeWeese J, Girardi M A, Sheppard V, Bodfish J W, N Engl J Med, 1999; 341: 1801-106.

Siegel J M, Cell, 1999; 98: 409-412.

Tooney P A, Crawter V C, Chahl L A, Biol Psychiatry, 2001; 49: 523-527.

U.S. Pat. No. 6,020,310 to Beck et al.

U.S. Pat. No. 6,020,314 to McMichael.

U.S. Pat. No. 6,197,746 to Beck et al.

U.S. Pat. No. 6,498,143 to Beck et al.

Zachrisson O, de Belleroche J, Wendt K R, Hirsch S, Lindefors N, Neuroreport, 1999; 10: 3265-3268.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method of treating one of schizophrenia and schizoaffective disorder in a patient in need thereof, the method comprising administering an effective amount of secretin to the patient.

2. The method of claim 1, wherein the secretin is administered by intravenous, oral, intramuscular, sublingual, intra-articular, transdermal, subcutaneous, inhalation, or rectal administration.

3. The method of claim 1, wherein the secretin is chosen from the group consisting of human secretin, porcine secretin, and bovine secretin.

4. The method of claim 1, wherein the secretin is synthetic secretin, natural secretin, or recombinant secretin.

5. The method of claim 1, wherein the secretin is administered as a single bolus.

6. The method of claim 1, wherein the effective amount of secretin is administered with a transdermal carrier.

7. The method of claim 6, wherein the transdermal carrier comprises dimethyl sulfoxide (DMSO).

8. The method of claim 6, wherein the effective amount of secretin and the transdermal carrier are mixed and applied to the skin together.

9. The method of claim 6, wherein the effective amount of secretin and the transdermal carrier are applied to the skin separately.

10. The method of claim 6, wherein the transdermal carrier comprises a gel or a lotion.

11. The method of claim 1, wherein the secretin is transdermally administered with a patch to be applied to a portion of the skin of the individual.

12. The method of claim 1, wherein the secretin is administered in an amount ranging from about 1 to about 500 clinical units.

13. The method of claim 1, wherein the secretin is administered in an amount ranging from about 2 to about 200 clinical units.

14. The method of claim 1, wherein the secretin is administered in an amount ranging from about 75 to about 150 clinical units.

15. The method of claim 1, wherein the schizophrenia is refractory schizophrenia.

16. A method for treating an individual exhibiting one or more symptoms of one of schizophrenia and schizoaffective disorder, the method comprising administering to the individual an effective amount of secretin, wherein one or more symptoms of one of schizophrenia and schizoaffective disorder are improved.

17. The method of claim 16, wherein the secretin is administered by intravenous, oral, sublingual, intramuscular, intra-articular, transdermal, subcutaneous, inhalation, or rectal administration.

18. The method of claim 16, wherein the secretin is chosen from the group consisting of human secretin, porcine secretin, and bovine secretin.

19. The method of claim 16, wherein the secretin is synthetic secretin, natural secretin, or recombinant secretin.

20. The method of claim 16, wherein the secretin is administered as a single bolus.

21. The method of claim 16, wherein the effective amount of secretin is administered with a transdermal carrier.

22. The method of claim 21, wherein the transdermal carrier comprises dimethyl sulfoxide (DMSO).

23. The method of claim 21, wherein the effective amount of secretin and the transdermal carrier are mixed and applied to the skin together.

24. The method of claim 21, wherein the effective amount of secretin and the transdermal carrier are applied to the skin separately.

25. The method of claim 21, wherein the transdermal carrier comprises a gel or a lotion.

26. The method of claim 16, wherein the secretin is transdermally administered with a patch to be applied to a portion of the skin of the individual.

27. The method of claim 16, wherein the secretin is administered in an amount ranging from about 1 to about 500 clinical units.

28. The method of claim 16, wherein the secretin is administered in an amount ranging from about 2 to about 200 clinical units.

29. The method of claim 16, wherein the secretin is administered in an amount ranging from about 75 to about 150 clinical units.

30. The method of claim 16, wherein the symptom is selected from the group consisting of positive symptoms, negative symptoms, affective symptoms, neurocognitive symptoms, social dysfunction, behavioral disorders and disorganized, compulsive, impulsive or repetitive behaviors.

31. The method of claim 30, wherein the positive symptoms are selected from the group consisting of active delusions, hallucinations, thought disorder, disorganized thinking and agitation.

32. The method of claim 30, wherein the negative symptoms are selected from the group consisting of lack of drive, lack of initiative, social withdrawal, apathy, emotional unresponsiveness impaired social relations, affective flattening, alogia and avolition.

33. The method of claim 16, wherein the schizophrenia is refractory schizophrenia.

34. A method of treating a schizophreniform disorder in a patient in need thereof, the method comprising administering an effective amount of secretin to the patient.

35. A method for treating an individual exhibiting one or more symptoms of a schizophreniform disorder, the method comprising administering to the individual an effective amount of secretin, wherein one or more symptoms of one of the schizophreniform disorder are improved.
